(19) United States
(12) Patent Application Publication
(10) Pub. No.: US 2023/0139687 A1
(43) Pub. Date: May 4, 2023

(54) Preparation Methods and Uses of Purified Fulvic Acid

(71) Applicant: Laiqun Zhang, Iowa City, IA (US)

(72) Inventor: Laiqun Zhang, Iowa City, IA (US)

(21) Appl. No.: 18/050,743

(22) Filed: Oct. 28, 2022

Related U.S. Application Data
(60) Provisional application No. 63/273,849, filed on Oct. 29, 2021.

(51) Int. Cl. C07D 493/04; C07B 63/00

(52) U.S. Cl. CPC C07D 493/04; C07B 63/00

(57) ABSTRACT

Fulvic acid prepared according to the inventions disclosed herein is suitable for human topical or oral application for hemostatic effect, for cosmetic effect, for antioxidative effect, for antiviral effect, or for elevation of immune system.

### BACKGROUND

Fulvic acid and humic acid a collective name of naturally occurring organic molecules existing in humic substances. Soil organic matter includes the remains of all plant and animal bodies which have fallen on the earth's surface or purposely applied by human. Humus is a brown to black complex variable of carbon containing compounds different from non humic substances (e.g. carbohydrates, fats, waxes, alkanes, peptides, amino acids, proteins, lipids, organic acids, etc.) The chemical formulae of humic substances cannot be readily and distinctively written. The non humic substances can be rapidly degraded by microorganisms in the soil, while humus is slow to decompose or degrade in the natural soil. Humus is the major soil organic matter component. Humic substances are the components of humus which are brown to black in color, hydrophilic, molecularly flexible polyelectrolytes. Three major categories of humic substances exist: Humin, Humic Acids, and Fulvic Acids.

Fulvic acid is the name of certain organic acids that are components of humic substances. Fulvic acid is lighter in color, mostly golden yellow, compared to Humic acids, brown or near black. Fulvic acid is also known to be of lighter molecular weight, more aqueous-soluble, and less polymerized than other component of the Humic substances such as Humic acids and Humin. Fulvic acids, typically but not exclusively, have molecular weights ranging from approximately 1,000 to 10,000. Fulvic Acids have relatively more carboxyl and hydroxyl groups and are more chemically reactive, and can readily enter plant roots, stems, and leaves.

Fulvic acids are known to be strong chelating agents, binding to metal ions. Humic substances, including the Fulvic acid, have been known to be applied as agricultural fertilizers. However, few attempts have been made to explore the suitability of Fulvic Acid to be applied to humans topically or orally. Nor have attempts been made to develop a method of production of fulvic acid with purity suitable for human use instead of agricultural use.

### DETAILED DESCRIPTION

According to one aspect of the current invention, purified Fulvic Acid is suitable for topical application to human wounds, punctures, and lacerations for hemostatic effect. Purified Fulvic Acid, when applied to human wounds, punctures, and lacerations in vivo, has shown significant hemostatic effect. Human subjects who have been wounded by metal sharps in accidents have been applied with purified Fulvic Acid solid powder produced according to the methods disclosed herein, and bleeding of wounds is reduced significantly within minutes.

According to one aspect of the current invention, purified Fulvic Acid is suitable for topical application to human skin as cosmetic active agent. Purified Fulvic Acid has shown antioxidative activity in vitro, and has shown appealing, natural colors when topically applied to human skin in vivo.

According to one aspect of the current invention, purified Fulvic Acid is suitable for human oral consumption. Purified Fulvic Acid, when fed to mice model up to oral intake amount limited by satiation, shows no toxicological effects. Purified Fulvic Acid produced according to the methods disclosed herein, when taken orally by human subjects, has shown anti-ageing effects, including reversing of hair-graying in human subjects.

According to one aspect of the current invention, purified Fulvic Acid has shown broad antiviral effects when applied topically or digested orally by a mouse model or a human subject.

According to one aspect of the current invention, purified Fulvic Acid has shown general elevation of immune system activities when applied topically or digested orally by a mouse model or by a human subject.

The method of producing purified Fulvic Acid is disclosed in details herein. Fulvic Acid may be initially prepared using biological fermentation or soil extraction. However, the preliminary products are only a mixture of various humic substances including humic acids and humin which are of undesirable, darker brown or black colors. Further, such preliminary products are tainted with unwanted ions such as Arsenic (As), Barium (Ba), Chromium (Cr), Mercury (Hg), Lead (Pb), Antimony (Sb), Selenium (Se), etc. Therefore, these preliminary products are not attractive in color and appearance and contain impurities, making them unfit for human oral intake or topical application.

Fulvic Acid is known to be a strong chelating agent, therefore conventional purification methods may not be sufficient to remove unwanted ions chelated to the Fulvic Acid preliminary products.

According to one aspect of the current invention, preliminary humic substances products are purified into fulvic acid with humic acids, humin, as well as impurity ions removed, where the end product has impurities at a level safe for human oral consumption or topical application and an attractive golden color for either topical application or oral consumption.

All values disclosed herein are approximate up to the last significant digit.

According to one aspect of the current invention, there is a method of purifying Fulvic Acid. Said method comprises:

Optionally, passing the preliminary biologically-produced product through a resin with affinity to Fulvic Acid;

Dissolving collected fulvic acid samples in water and adjusting to a slightly acidic pH of 4.0-5.0;

Centrifuging the solution and discarding the pellet;

Washing a resin with affinity to heavy metal ions with water;

Passing the supernatant solution through the washed resin;

Discarding the initial passthrough solution with basic (higher than 8.0) pH and collecting the later passthrough solution with neutral or acidic (no higher than 7.5) pH; and

Optionally drying the collected solution.

One illustrative non-limiting example of the method according to the current invention is disclosed here. For larger batches of production the amount can be adjusted proportionally, and the specific brands of the resin and equipment can also be altered according to the knowledge of the skilled artisan in the fields of organic chemistry, biochemistry, and chemical engineering.

The method is:

1. Dissolving 100 g unpurified Fulvic Acid in water to a volume of 500 mL and adjusted to a pH of 5.0.
2. Centrifuging the solution for 30 minutes at 12000 g and discarding the pellet;
3. Soaking 50 g of FerrIXA33E in water, leaving it standing.
4. Loading the resulting FerrIXA33E onto a 50 mL volume resin column and washing the resin column with 400 mL of water;
5. Loading the supernatant solution from step 2 into the resin column, discarding the initial passthrough which has a pH of about 9.0 and collecting the passthrough liquid when the pH of the passthrough decreases to a pH of 7.0 or lower;
6. Drying the collected passthrough solution.

A sample of unpurified good-looking (mostly yellow in color) Fulvic Acid purchased over the open marketplace is purified according to the method disclosed above. The purchased unpurified sample, the Step 2 centrifuge supernatant, and the final passthrough have been tested for impurity ions, utilizing ICP atomic emission technique per ASTM E1479-16, minimum detection threshold 1 ppm (parts-per-million). Impurity ions are significantly reduced. As examples, FIG. 1 lists select impurities present in the purchased unpurified sample have been reduced or removed. A skilled artisan would have understood that this method removes other contaminants in addition to Sb, Se, Cr, and Ba from the unpurified Fulvic Acid.

FIG. 1 Impurity Test Results (ppm)

| Sample | Total Sb | Total Se | Total Cr | Total Ba |
|---|---|---|---|---|
| purchased | 2 | 1 | 1 | 4 |
| unpurified sample | | | | |
| centrifuge | 1 | nil | nil | 2 |
| supernatant | | | | |
| final passthrough | nil | nil | nil | 2 |

*nil: below detection threshold of 1 ppm.

What is claimed is:

1. A method of purifying Fulvic Acid, comprising adjusting an aqueous solution of a fulvic acid sample to pH 4.0-5.0; centrifuging said solution, passing a supernatant of said centrifuging through a resin; discarding an initial pass through solution that is basic; collecting a later pass through solution that is neutral or acidic.

2. A method of purifying Fulvic Acid according to claim 1, wherein said resin has affinity to heavy metal.

3. A method of purifying Fulvic Acid according to claim 2, wherein said initial pass through solution has a pH value of at least 8.0.

4. A method of purifying Fulvic Acid according to claim 2, wherein said later pass through solution has a pH value of at least 7.5.

5. A method of purifying Fulvic Acid according to claim 2, wherein said resin is a resin column loaded with FerrIXA33E.

6. A method of purifying Fulvic Acid according to claim 1, wherein said aqueous solution of fulvic acid sample is adjusted to a pH of 5.0.

7. A method of purifying Fulvic Acid according to claim 2, wherein said later pass through solution has lower than 1 ppm of each of Sb, Se, and Cr impurities.

8. A method of purifying Fulvic Acid according to claim 2, wherein said later pass through solution has no higher than 2 ppm of Ba impurities.

9. A composition for topical application, comprising Fulvic Acid.

10. A composition for oral ingestion, comprising Fulvic Acid.
